(19) United States
(12) Patent Application Publication
(10) Pub. No.: US 2013/0330244 A1
(43) Pub. Date: Dec. 12, 2013

(54) COMPOSITIONS AND METHODS FOR TOPICAL NITRIC OXIDE GENERATION

(71) Applicant: NIOXX LLC, (US)

(72) Inventors: Alexandru T. Balaban, Galveston, TX (US); William A. Seitz, Dickinson, TX (US); Kenneth A. Smith, Houston, TX (US)

(73) Assignee: NIOXX LLC, Dickinson, TX (US)

(21) Appl. No.: 13/688,511

(22) Filed: Nov. 29, 2012

Related U.S. Application Data
(60) Provisional application No. 61/566,934, filed on Dec. 5, 2011.

(51) Int. Cl. A61M 35/00

(52) U.S. Cl. CPC A61M 35/003
USPC 422/225; 422/129; 422/236

(57) ABSTRACT

A simple, biocompatible two-component system and procedure for generating nitric oxide (NO) is described. One component comprises sodium nitrite or other nitrite source, and the other component comprises a reductant, an acid and a base although in certain embodiments the reductant and acid functions are provided by the same component. When these two components are mixed directly at a local site of administration or immediately prior to application and the mixture generates nitric oxide (NO) for topical application. The activated system is therapeutic for treatment of multiple conditions, including promotion of healing, disinfection, promotion of hair growth, and treatment of male and female sexual dysfunction.

### CROSS REFERENCE TO RELATED APPLICATIONS

This application claims priority based on U.S. Provisional Application Ser. No. 61/566,934 filed Dec. 5, 2011, which is incorporated herein by reference.

### FIELD OF THE INVENTION

This invention relates compositions and methods for generating nitric oxide locally.

### BACKGROUND OF THE INVENTION

Without limiting the scope of the invention, its background is described in connection with application of nitric oxide in medical indications and compositions and methods for nitric oxide (NO) generation locally.

The biological importance of NO is well documented (Lancaster J R, Proc Natl Acad Sci (1996) 91: 8137-41; Ignarro et al, Proc Natl Acad Sci (1987) 84: 9265-69; reviewed in Bredt D S, J Cell Science (2003) 116: 9-15; reviewed in Murad F, N Engl J Med (2006) 355: 2003-11.). In mammals, NO is an endogenous physiological mediator of many processes in the nervous, immune and cardiovascular systems. These include vascular smooth muscle relaxation, which results in arterial vasodilation and increased blood flow. NO is also a neurotransmitter and has been associated with neuronal activity and various functions like avoidance learning. NO also partially mediates macrophage cytotoxicity against microbes and tumor cells. Besides mediating normal functions, NO is implicated in pathophysiologic states as diverse as septic shock, hypertension, stroke, and neurodegenerative diseases.

NO has been applied pharmacologically in various forms. See Butler and Feelisch, Circulation (2008) 117:2151-59. Inorganic nitrates acting as NO donors such as nitroglycerin and sodium nitroprusside have been long used to correct NO deficient states or to regulate the activities of many tissues. Topical applications may be used to help wound and burn healing, hair growth, impotence, and to cause vasodilatation where needed (e.g., ripening of the cervix in pregnancy). Local high concentrations of NO (eye, skin, e.g.) are tolerated. Smith et al. (U.S. Pat. No. 5,519,020) describes polymeric nitric oxide sources thought to be useful to promote healing.

Two types of NO synthases (inducible and constitutive) produce NO in living organisms from L-arginine Delivery of L-arginine for increased production of NO is well known, including by topical delivery for erectile dysfunction and hair growth such as disclosed by Fossel in U.S. Pat. No. 7,914,814.

Synthetic NO donors are also of two different types: those that spontaneously evolve NO from chemical precursors, and, those that need metabolic redox processes for releasing NO. See Scatena et al. Current Medicinal Chemistry (2010) 17:61-73. The first type of synthetic NO donor includes chemical precursors that release NO slowly in an aqueous environment that is slightly acidic, neutral or slightly basic. These include those that employ N₂O₂ groups such as are disclosed by Smith et al. in U.S. Pat. No. 5,691,423 and related patents; polyamine/NO adducts as in Hrabie and Keefer U.S. Pat. No. 5,683,668; a broad class of polyamine-derivatized diazeniumdiolates which were extensively reinvestigated by Keefer and co-workers, as described in a number of U.S. Patents including for example U.S. Pat. No. 7,226,586; other NONOates such as in Smith et al U.S. Pat. No. 6,147,068, and N-nitroso-N-substituted hydroxylamines such as in Garfield et al U.S. Pat. No. 5,698,738.

In U.S. Patent Application Serial No. 2009/0081279, Jenek disclosed a wound dressing in which the NO donor S-nitrosoglutathione was generated in situ by diffusion between a polymer containing an S-nitrosothiol overlaid by a polymer containing a nitrite salt. Generation of S-nitrosoglutathione was slowed and controlled by buffering. In a subsequent application, U.S. Patent Application Serial no. 2011/0070318, Jenek disclosed use of non-thiol non-acid reducing agents such as the exemplified hydroquinone to limit production of nitrogen dioxide from mixtures of acidified nitrites. In U.S. Patent Application Serial No. 2003/0039697, Zhao et al similarly described a generation of NO in a matrix by interaction between the reducible NO donors S-nitrosoglutathione or nitroprusside by interaction with a reductant such as ascorbic acid. The synthetic NO donors that require metabolic redox processes for releasing NO (usually from higher oxidation states) or stimulate endogenous production of NO include organic nitrates such as glycerol trinitrate (trivially known as nitroglycerin) and isosorbide dinitrate, which have been long used in medicine, but are known to produce tolerance, i.e., the need to progressively increase the dose in order to obtain a constant effect. These are also known to produce undesirable systemic side effects (e.g., headache) although topical formulations have been described such as by Russell (U.S. Pat. No. 6,287,601). Other synthetic NO donors falling in this second category include some amidine derivatives such as those disclosed in Currie et al U.S. Pat. No. 5,674,894 and 3-(nitrooxymethyl)phenyl 2-hydroxybenzoate (referred to as B-NOD) such as disclosed by Bing in U.S. Pat. No. 6,538,033. Also included would be nitric oxide synthase stimulators, which are described in a number of US Patents including for example in Strobel et al U.S. Pat. No. 7,179,839.

Nitric oxide is present naturally in the atmosphere at concentrations of 10 to 100 parts per billion. Because NO is a product of combustion, NO is present in exhaled tobacco smoke at concentrations of 400 to 1,000 parts per million (ppm). Therapeutically, iNO has been administered at a dosage typically between 20 and 40 parts per million in air to humans having breathing problems and has beneficial effects due to its bronchodilatory and vasodilatory activity. In 1999, the FDA approved inhaled NO (iNO) for treatment of hypoxic respiratory failure in near-term infants and newborns for whom conventional ventilator treatment has failed. iNO has been administered to adults off label for treatment of pulmonary arterial hypertension and adult right-sided heart failure among other diseases.

However, the colorless gaseous NO may (under some conditions) react rapidly with atmospheric oxygen, yielding nitrogen dioxide (NO₂), a red-brown gas with much higher toxicity than NO. Furthermore, when administered at high levels (>80 ppm) for prolonged periods, iNO converts oxygen-carrying hemoglobin into methemoglobin, which may lead to impaired tissue delivery of oxygen. See, e.g. B Weinberger, et al. The Toxicology of Inhaled Nitric Oxide, Toxicological Sciences 59 (2001) 5-16.

Nitrous acid (pKₐ=3.37) is produced from inorganic nitrites on treatment with acids (HA). Nitrous acid is stable in aqueous solution at low temperature, but it decomposes into NO and NO₂ readily at room temperature according to the equations (1) and (2):

2HA+2NaNO₂→2HNO₂+2NaA (1)

2HNO₂→NO+NO₂+H₂O (2)

Reaction (1) above was modified to introduce the sodium nitrite to a buffer solution to generate low concentrations (<100 ppm) of gaseous nitric oxide entrained in a flow of delivery gas for inhalation treatment of pulmonary hypertension after further purification of the released gas to remove toxic nitrous acid and nitrogen dioxide. See Fine et al. U.S. Pat. No. 7,040,313.

Under current U.S. Department of Agriculture regulations, the specification for curing meat (especially bacon) and imparting a pink color to it using nitrites, sodium or potassium nitrite is used in combination with reducing agents such as sodium ascorbate or sodium erythrobate to accelerate the curing process and reduce the ultimate levels of nitrite in the meat (9 CFR Vol 2, Subpart C, §424.21; Mirvish SS, Toxicol Appl Pharmacol (1975) 31:325-51; McCutchen JW. Public Health Reports (1984) 99:360-64). However, the above-mentioned uses antedate considerably the discovery of NO as an important physiological mediator, and until now the methods and procedures selected by the inventors are not described as a means for topical delivery of nitric oxide.

Nitric Oxide (NO) plays a role in the physiological manifestation and treatment of conditions that include:
Cardiovascular: hypertension; angina; atherosclerosis; preeclampsia (pregnancy induced hypertension); toxemia; eclampsia; HELP syndrome; regulation of vascular conductance; regulation of blood flow; regulation of blood pressure; myocardial ischemia, blood clotting, and restenosis following implant of stents.
Cancer treatment: controlling oxidative damage in conjunction with chemotherapy and radiation treatment.
Gastrointestinal: altered motility; and pyloric stenosis.
Lung Function: asthma; treatment of premature babies to increase lung function; and pulmonary hypertension.
Inflammation: autoimmune and immune diseases; acute inflammation; arthritis; resistance to infection; cancer; SLE-Lupus; anaphylactic reactions; and allograft rejection.
Central Nervous System: behavior; epilepsy; Alzheimer's disease; stroke; and growth hormone disorders (e.g., acromegaly).
Pancreas: diabetes.
Female Reproductive System: ovulation; female sexual arousal disorder; implantation/in vitro fertilization; premenstrual syndrome; dysmenorrhea; uterine contractile disorders; premature labor; cervical dilation; contraception; menopause symptoms; osteoporosis; endocrine disorders; and hormone replacement therapy.
Male Reproductive System: impotence; erectile dysfunction; male menopause symptoms; endocrine disorders; osteoporosis; and prostate hypertrophy.
Bladder and Kidney: incontinence; renal arterial stenosis; and hypertension.
Skin^{.} eczema (skin reaction to foreign particle); autoimmune skin diseases; topical hair loss; acne; wounds; and burns.
Antibacterial: NO is a potent microbicide and can be effective against antibiotic-resistant microbes.

The present invention includes formulations and methods for treating the above listed systems and conditions through local application of an acidified nitrite composition that directly releases NO gas in situ.

### SUMMARY OF THE INVENTION

The present invention provides an acidified nitrite composition for local pharmaceutical application of effective amounts of NO at values of pH that are acceptable from regulatory and medical standpoints. The present inventors have surprisingly found that acidified nitrite compositions comprising a base, preferably a conjugate base of the acid used to acidify the composition, can produce substantial amounts of NO at pH values higher than 4. The NO producing acidified nitrite admixture of this invention comprises nitrite, a reductant, a mild acid and a base. The preferred sources of nitrite and base are soluble salts. In one embodiment, these components are combined in a diffusion-inhibiting medium which controls the rate of nitric oxide release and is sufficiently viscous to apply topically.

Various nitrite salts may be used, most commonly inorganic ones such as sodium nitrite, although potassium nitrite, calcium nitrite, or any alkali or alkali earth nitrite should be usable. The preferred reductant is one having the reductive capability of preventing or slowing the oxidation of nitric oxide (NO) to nitrogen dioxide (NO₂), and also having the capability of directly reducing NO₂ to NO so that the gas released by the composition is predominantly NO. Suitable reductants include ascorbic acid and its derivatives, ascorbate salts, tocopherols (including particularly alpha tocopherol), erythrobates, carotenoids, tocotrienols and thiols.

Citric acid is one acceptable organic acid. Other acids may include lactic acid, glyceric acid, formic acid or other organic acids known to those of skill in the art. Inorganic acids with the appropriate pKₐ values can also be used if they are biologically acceptable (e.g. boric acid). The base is preferably the conjugate base of the acid used, but can be another organic or inorganic base known to those of skill in the art. The medium for dissolution of the nitrite, acid, reductant and base may be an aqueous medium or, in fact, a nonaqueous medium. The medium functions as a vehicle for application of the composition and also serves to reduce the rate of the chemical reaction producing NO. Aqueous media are generally preferred and readily prepared as gels, although organic salves, or inorganic preparations such a petroleum jelly are also suitable.

In another embodiment an acidified nitrite gel is provided in which ascorbic acid, or an ascorbic acid derivative, serves both as the organic acid and the reductant. Ascorbic acid (vitamin C) is one biocompatible reducing agent for nitrites, and its derivatives include, but are not limited to, 3-O-ethyl ascorbic acid, other 3-alkyl ascorbic acids, 6-O-octanoyl-ascorbic acid, 6-O-dodecanoyl-ascorbic acid, 6-O-tetradecanoyl-ascorbic acid, 6-O-octadecanoyl-ascorbic acid, and 6-O-dodecanedioyl-ascorbic acid.

The present invention, in one important aspect, involves a composition for generating and controlling the release rate of nitric oxide for topical applications that involve more than one gel or, or alternatively, another viscous vehicle. In one embodiment, the first aqueous gel comprises nitrite and a second aqueous gel comprises an acid and a base to modify the pH of the gel. A reductant to help retain the nitric oxide in bioactive form is included in the first or second gel. Where ascorbic acid alone is used, the ascorbic acid can function as both the acid and the reducing agent. However, if an acid such as ascorbic acid is the reductant, it is included in the second gel to avoid initiating production of NO₂. In certain embodiments, the base is a conjugate base of the acid, which may be provided by dissolution of a salt of the acid used. Thus, one biocompatible embodiment utilizes citric acid as the acid, sodium citrate to provide the citrate ion base, and further includes ascorbic acid which functions as a further proton donor (acid) and as a reductant. Where ascorbic acid is used as the acid and reductant, the preferable salt is sodium ascorbate. When the first and second aqueous gels are mixed together, nitrite is chemically converted to nitric oxide.

Gellification agents include substances such as hydroxymethylcellulose hydroxyethylcellulose, gelatin, agar, natural gums, starches and pectins, for example.

In another embodiment, the first and second gels may be combined in layers with the nitrite-containing gel preferably in contact with skin. Prior to application these gels can be separated by an impermeable plastic or metal foil if desired. They can be applied directly to the skin or with an interposed gas-permeable membrane present to avoid possible skin irritation.

The topical application includes, of course, application to the skin. Other local applications are envisioned including intracavitary applications. A mixture of powdered sodium nitrite, ascorbic acid, (or other reductant) and citric acid (or another organic acid of adequate strength) and a base-producing salt immediately generates nitric oxide (NO) upon addition of water. To slow the NO generation, one may prepare an ointment from a nonaqueous medium (petrolatum, Vaseline, e.g.) and the four powdered ingredients (nitrite, acid, base and reductant), which, on being applied topically on the skin, will release NO as water permeates through this medium. Alternatively, one may convert the aqueous sodium nitrite solution into an aqueous gel with hydroxyethylcellulose (or other gel-forming substance or compound) and combine this gel with another gel obtained from aqueous ascorbic and citric acids and hydroxyethylcellulose for topical application (on intact skin, burns, intra-cavity, etc.). The two gels may be admixed immediately before use or may be applied in sandwich-like fashion (possibly as a transdermal patch) for further slowing down the delivery of NO.

In another embodiment, NO is therapeutically applied by a method comprising combining a nitrite salt, a biocompatible reductant, an acid, and a base in a medium and topically applying the combination to a body site. This method for the topical delivery of nitric oxide is accomplished by steps comprising providing a powdered nitrite salt with a diffusion-inhibiting, topically applicable medium and mixing this with a powdered reductant, an acid and a base-producing salt in a second diffusion-inhibiting, topically applicable medium. The medium is then applied in an effective amount to a desired body site. The diffusion-inhibiting characteristic of the medium slows and controls the reactions among the nitrite, acid and base so as to prolong the release of nitric oxide. Such a composition that provides controlled release of nitric oxide is helpful in topical application, for example, to the skin or other body surface. Methods for the application of these materials to a desired area are many fold, some of which are mentioned here and include applying a nitrite-containing gel or salve layer to the skin or other local body site. This would be followed by overlaying a layer comprising acid, reductant and base. This should give rise to a controlled rate of nitric oxide release to contact the desired bodily surface. These can be manually applied or can be applied as premeasured layers. In some cases the gels may simply be mixed just prior to application to form a relative homogeneous but diffusion-inhibiting salve or gel with all components mixed therein and a sufficiently slow and controlled rate of nitric oxide generation.

Aqueous gelling agents usable in the methods of the present invention include agars, hydroxyethyl celluloses and many other materials known to those of skill in the art usable in preparing aqueous-based gels. The appropriate gels containing the active ingredients outlined herein may be prepared in advance and packaged separated by an impermeable plastic or metal layer, meant to be removed just before use. After removal the layers may be topically applied, the nitrite-containing layer being preferably applied closest to the body site. In some cases, it may be desirable to interpose a gas permeable membrane on the body site prior to the application of the gel or ointment nitric oxide source. This may lessen any skin irritation possibly resulting with certain individuals.

In certain embodiments, a two-component nitric oxide (NO) delivery system is provided that includes a first chamber including a plunger and containing a nitric oxide donor composition comprising a nitrite salt in a first gel, and a second chamber including a plunger and containing a nitric oxide activation composition comprising at least one reductant, at least one organic acid and at least one conjugate base of the organic acid mixed together in a second gel. The first chamber and the second chambers are adapted store the nitric oxide donor and activation compositions separately until nitric oxide generation is desired at which time the plungers are adapted to be moved synchronously into the first and second chambers and thereby extrude measured amounts of the nitric oxide donor and activation compositions into a mixing chamber that is adapted to admix the measured amounts and extrude a nitric oxide generating admixture to a tissue site. In certain embodiments the pH of the nitric oxide activation composition is greater than 3.0 and the admixture is characterized by an initial pH greater than 4.0. The chambers of the two-component nitric oxide (NO) delivery system may be preloaded with a plurality of single doses and in certain embodiments the chambers are marked with single dosage markings that indicate each single dosage amount to be delivered with each depression of the plungers. If desired the doses may be administered to a tissue site repeatedly to provide extended exposure to effective amounts of NO. This may be particularly desired in wound healing and hair growth indications. An occlusive bandage is packaged together with the nitric oxide donor and activation compositions disposed in the first and second chambers in certain embodiments.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1A shows the quantity and time dependence of NO evolution from gel mixtures made with different amounts of sodium citrate. The gel compositions are shown in Table 1, and they are identified on the figure by the amounts of sodium citrate they contain in weight percent. The curves are all plotted in the same arbitrary units.

FIG. 1B shows the evolution of the measured volume of foam generated by the mixed acid and nitrite gels in the measurement procedure of Example 3.1. The data shown are for a mixture containing Gel 4 in Table 2, which has 10% sodium citrate by weight. The acid and nitrite gels are mixed within a 2-second period as they are being injected at time designated zero (0) into a graduated cylinder.

FIG. 2A shows relative NO release for the different gel mixtures as determined by integration of the curves shown in FIG. 1A from time t=0 to a time of 20 minutes. The gel compositions are shown it Table 1, and they are identified on the figure by the amounts of sodium citrate they contain in weight percent. The curves are all plotted in the same arbitrary units.

FIG. 2B shows NO release as determined by the measurement procedure of Example 3.1 for different mixtures of acid gel and nitrite gel. The acid gel compositions used in the mixtures are shown in Table 2, and are identified on the figure by the amounts of sodium citrate they contain in weight percent. The vertical axis shows the volume of NO gas release per ml of nitrite gel in the mixture.

FIG. 3 shows measured blood perfusion in test areas of skin with and without NO in locally applied gels.

FIG. 4A depicts an embodiment of a NO generating system that utilizes a double barreled mixing syringe and a static mixer nozzle. FIG. 4B depicts an embodiment of an NO generating system where the nitric oxide donor and activation compositions are supplied separately from premeasured containers and applied over a tissue site in need thereof.

### DESCRIPTION OF ILLUSTRATIVE EMBODIMENTS

U.S. Pat. No. 6,103,275 taught generation of nitric oxide locally by a two-component system wherein an acidified nitrite composition was generated by mixing a nitrite salt with an unbuffered acid, specifically maleic acid having a pKa between of 1.83, together with ascorbic acid (pKa 4.1) as a reductant. The present disclosure provides an effective two-component system that generates NO at surprisingly higher pH values that are more physiologic, less irritating and thus more desirable in certain indications.

In the present disclosure, a simple procedure is provided for generating nitric oxide (NO) from water or biological fluids and a mixture of biocompatible reagents, and also for converting aqueous solutions of such reagents into ointments or gels for topical application and slow delivery of NO. However, with a composition comprising reducing agents, NO is the predominant product, this constituting one of the aspects of the present invention.

Biocompatible systems and procedures for generating nitric oxide (NO) are described herein that are particularly useful for topical applications.

The methods described herein include mixing an aqueous solution of sodium nitrite with an aqueous solution of an equimolar amount of citric acid, in the presence of an excess of ascorbic acid and an amount of sodium citrate to increase the pH of the solution. The sodium nitrite is preferably kept separately from the acidic ingredients. Advantages of the present procedure in one preferred embodiment include employing three safe and inexpensive compounds with convenient characteristics for producing NO free of any other residue that may cause adverse biological effects (as is potentially the case of many synthesized NO donors), and also free from the need of enzymatic reactions that may lead to tolerance (as is the case of organic nitrates functioning as NO donors).

It was previously believed that an acidified nitrite composition for therapeutic topical application would only release NO if the mixture had a pH less than 4. For example in U.S. Pat. No. 6,709,681, NO production was drastically reduced below pH 3 and no substantial amount of NO was detected with a mixture of sodium nitrite and an acid buffered to pH values over 4. Similarly in Jezek U.S. Patent Application Serial no. 2011/0070318, an activating low pH is achieved with use of hydrochloric acid. The '318 application specifies the use of non-thiol reducing agents that are not acids. The buffering action of nitrite is utilized to maintain a preferable pH from 3 to 4. While such low pH compositions do release NO, the low pH can be too irritating thus working against the therapeutic effect.

As disclosed herein, improved mixtures are provided based on the discovery that an acidified nitrite gel constituted as described herein can produce substantial amounts of NO at pH values above 4.0. Additionally, the inventors are not aware of compositions reported, marketed or used that consist of: (i) a nitrite salt with (ii) an organic acid of adequate strength, (iii) a reducing agent, and (iv) a base-producing salt, all four ingredients being biocompatible. Few organic or other acids of the necessary strength are biologically tolerated, and in the absence of a reducing agent such as ascorbic acid, the deleterious nitrogen dioxide could also be produced along with NO, as seen in eq. (2). One may therefore assert that the method and procedure described herein are unique in terms of the necessity of having the four types of ingredients selected by the inventors, and among representatives of these types the inventors have selected preferred ones.

One reaction scheme through which this takes place is:

2HA+2NaNO₂→2HNO₂+2NaA (1), where HA is an organic acid such as citric acid

2HNO₂→NO+NO₂+H₂O (2),

nitrous acid decomposes generating nitrogen dioxide

NO+NO₂+H₂O+Asc(OH)₂→2NO+2H₂O+AscO₂ (3),

where the ascorbic acid reacts to remove the nitrogen dioxide formed

Through reactions (1) and (2), H⁺ is removed from solution, and the pH of the mixture increases as the reaction proceeds. Therefore, the initial pH of the mixture observed immediately after mixing is the lowest pH observed.

A two gel method for delivery of NO will have the following properties. Dosage (total) can be controlled simply by adjusting the quantity of nitrite and acid. Rate of NO release can be independently controlled by adjusting the viscosity of the gel. Thus, a high total dosage can be delivered over a long period of time or a low total dosage can be delivered rapidly, as desired. In addition, various physical means for applying successive doses can be easily developed. For example, multilayer sandwiches could be formed with each successive layer activated by removing sequential barriers between gels (which themselves could even be of different strengths). Thus, a wound could remain covered for several treatments. Another feature of the gels is that they are compatible with the addition of various agents such as sterilizing compounds and antibiotics.

Other mechanisms of application (other than topical) are possible. This technology might be used as sprays, suppositories, (aural, nasal, vaginal or rectal) or even injectable form to control many biological functions. It might also be dispensed in dropper form to be used in the eye, ear, nose or throat. Almost all these applications deal with treatment of inflammation. The intravenous applications might be useful for acute angina and to regulate the cardiovascular system.

The gel might also be used in combination with various agents including antibiotics, anesthetics, analgesics, anti-inflammatory agents such as corticosteroids and nonsteroidal anti-inflammatory agents, antiviral agents, vasodilators or vaso-constrictors, sunscreen preparations (PABA), antihistamines, other hormones, such as estrogens, progesterone, androgens, antiseborretic agents, other cardiovascular agents, mast cell stabilizers, scabicides or pediculicides, keratolytics, lubricants, narcotics, shampoos, acne preparations, antiseborrheic agents, burn preparations, cleansing agents, deodorants, depigmenting agents, diaper rash products, emollients and moisturizers, photosensitizing agents, poison ivy or poison oak or sumac products, sunburn preparations, tar-containing preparations, wart preparations, wet dressings and wound care products. This would reduce any potential danger of infection introduced by the process.

The present gel technology is preferably but not necessarily a local NO delivery system as opposed to a systemic one. Therefore, the many systemic side effects of other NO treatments (such as nitroglycerin) should be completely avoided. This is an important advantage for certain indications.

A further advantage of the local application of the gels is that it is self-regulating in the sense that when the desired effect of the treatment has been achieved, the remaining amount can simply be wiped off and the NO release stops. For subjects who might have some allergic response to other treatments, this ability to immediately stop treatment should be beneficial.

In addition to the list of uses and potential uses of NO, four important specific applications of the gel technology are worthy of more detailed discussion. These are generally related to the fact that the gels produce NO locally which in turn enhances local circulatory response.

1. Since topical application of the gels has been shown to immediately enhance local blood flow, the gel technology can have important uses in treatments of male and female sexual and reproductive problems, especially penile and clitoral erection and impotence. The ability to control dosage directly may be important here. Of course, if desired, antibiotics, spermacides and/or other additives may be included in such a gel.

2. Enhancement of local circulation is important for hair replacement and growth. The gel technology can be used to treat topical hair loss, particularly insofar as the hair loss is at least partially caused by microcirculation defects.

3. Burns respond to treatment with locally applied NO as demonstrated in animal models. See Zhu et al. J Burn Care & Research (2008) 29:804-14. Use of the gel technology in conjunction with other compounds may have application to even minor burns such as sunburn and other wounds.

4. NO donor compounds are important in the control of cervical dilation. The gel technology for this purpose is particularly appealing. First, it can be controlled directly and second, it is purely local as opposed to systemic.

There are numerous types of compounds which might be added to gels without unfavorably altering the NO donation properties but which would have some added features already known. These include antibiotics, steroids, antihistamines, anti-infective agents, prostaglandins, antipyretics, analgesics, antiseborrheic agents, anti-psoriasis agents, antipruritics and local anesthetics. Also they may be combined with locally acting cardiovascular agents, e.g., alpha or beta blockers and Rogaine. Another type of compound that might be added to the gels is a vasodilator such as the alpha adrenergic receptor antagonist phentolamine mesylate or other sexual erectile agents. One can also combine the gels with vitamins, skin softeners, emollients, clearing agents, enzymes and keratolytics.

The following examples are included to demonstrate preferred embodiments of the invention. It should be appreciated by those of skill in the art that the techniques disclosed in the examples which follow represent techniques discovered to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Preparation of Acidified Nitrite Gel for Topical Release of Nitric oxide

The gels containing nitrite and acid are mixed individually and maintained separately until use. To prepare the nitrite gel, 85 ml deionized water was heated to 80° C. Two grams (2 g) of sodium nitrite was added, stirring until completely dissolved. Next 2.88 g of hydroxyethylcellulose (HEC) having an average molecular weight of 750,000 was added. The mixture was stirred until the HEC was completely gelled and then allowed to cool below 45° C. Deionized water was added to make up to a total product weight of 100 g, and stirred to ensure good mixing.

To prepare the acid gel, 70 ml deionized water was heated to 80° C. To this was added 5.6 g citric acid, 5.2 g of ascorbic acid (vitamin C) and, optionally, a measured amount of sodium citrate, with stirring until all components were dissolved. In certain embodiments, the ascorbic acid derivative 3-O-ethyl ascorbic acid was used. Then, 2.88 g of hydroxyethylcellulose (HEC) having an average molecular weight of 750,000 was added. The mixture was stirred until the HEC was completely gelled and then allowed to cool below 45° C. Deionized water was added to make up to a total product weight of 100 g, and stirred to ensure good mixing.

On admixing equal amounts of the two gels immediately before use, placing the mixture on intact skin, and covering it (or not) with an adhesive bandage, NO will be delivered topically. It is preferable (when mixing the two gels is not done, or is done on the skin) to have the gel with nitrite in contact with the skin, and to apply the other gel over it, in order to reduce any irritation due to the low pH of the mixture or acid gel. If the two gels have sufficient consistency, thin slices of appropriate dimensions from each of the two gels can be cut and sandwiched, separated by an impermeable plastic or metal foil; immediately before use the foil can be removed, the two slices can be slightly pressed against one another and covered by the air-tight adhesive bandage, if desired, for gradual topical delivery of NO.

Contact between the atmosphere and the mixture producing nitric oxide is preferably avoided or minimized because, under some conditions, NO may be rapidly oxidized by air (unless the NO is extremely dilute) to afford undesirable nitrogen dioxide.

### EXAMPLE 2

### Preparation pH-Controlled Gel Mixtures

Following the above mixing procedure of Example 1, four citric acid-containing gels were prepared with different amounts of sodium citrate. The weight percentages of sodium citrate and the molarity of the primary components are shown in Table 1 for these four gels. Each gel had molarities of 0.30M citric acid, 0.28M ascorbic acid and 4.4×10⁻⁵M hydroxyethylcellulose of molecular weight 750,000. The citric-acid based gels including various amounts of sodium citrate were admixed with an equal volume of a nitrite containing gel having molarities of 0.32M sodium nitrite and 4.4×10⁻⁵M hydroxyethylcellulose of molecular weight 750,000 to produce NO. The initial pH values of the mixtures of the acid and nitrite gels are also shown in Table 1. Increasing the amount of sodium citrate increases the pH of the acid-containing gels themselves and as well as the initial pH of mixtures of the acid-containing gels and the nitrite-containing gels.

TABLE 1

| | | | | | Maleic |
|---|---|---|---|---|---|
| | Gel 1 | Gel 2 | Gel 3 | Gel 4 | acid gel |
| Sodium citrate content, | 0% | 1% | 3% | 10% | 0% |
| wt % | | | | | |
| Citric Acid | 0.30M | 0.30M | 0.30M | 0.30M | |
| Sodium Citrate | 0M | 0.04M | 0.11M | 0.38M | |
| Ascorbic Acid | 0.28M | 0.28M | 0.28M | 0.28M | |
| 3-O-Ethyl Ascorbic Acid | | | | | 0.25M |
| Maleic acid | | | | | 0.29M |
| pH of Acid-containing | 2.11 | 2.5 | 3.28 | 4.45 | 1.77 |
| gel | | | | | |
| Initial pH of Gel mixture | 3.41 | 3.9 | 4.25 | 4.97 | 2.50 |
| incl. nitrite | | | | | |

Addition of sodium citrate to the acid gel provides additional A—in the acid dissociation reaction (shown in EXAMPLE 2.1 below) and shifts the acid dissociation equilibrium so that the H+ concentration is reduced, increasing the pH of the acid gel. It was initially thought that high pH mixtures would not release sufficient amounts of NO. In U.S. Pat. No. 6,103,275 generation of nitric oxide locally by a two-component system resulted from an acidified nitrite composition using an unbuffered maleic acid, which has a pKa of 1.83. As shown above, when a maleic acid composition is prepared that is comparable to those using buffered citric acid, the initial pH of the gel mixture including the nitrite is 2.5. The present inventors' work, as shown above, shows that the addition of 10% sodium citrate reduces the amount of NO released by no more than a factor of 2 but increases the pH of the mixed product from 3.41 to 4.97, which is a reduction in its acidity by a factor of 35. This provides a significant improvement to the suitability of acidified NO producing admixtures for therapeutic indications.

### EXAMPLE 2.1

### Preparation pH-Controlled Gel Mixtures

In another example, following the mixing procedure of Example 1, five acid gels were prepared with different amounts of sodium citrate. The weight percentages of sodium citrate and the molarity of the active components are shown in Table 2 for these gels. Table 2 also shows the pH of each acid gel, as measured after its preparation. A nitrite gel was prepared following the mixing procedure of Example 1, which provides a nitrite gel having molarities of 0.29M sodium nitrite and 3.84×10⁻⁵M hydroxyethylcellulose of molecular weight 750,000. Approximately 1.5 ml of the first acid gel and an equal amount of nitrite gel were respectively loaded into opposite cylinders of a dual-cylinder, 2 ml mixing syringe fitted with a static mixer (Syringe 4B19 with a 4.6 mm×16 element static mixer, both from Plas-pak Industries, Norwich Conn., USA). Operating the mixing syringe causes the gels to mix as they flow through the static mixer. The contents of the mixing syringe are rapidly and thoroughly mixed in about two seconds as they flow through the static mixer. A mixed gel from which gaseous NO is evolving flows out of the static mixer. The pH of the mixed gel was measured immediately after mixing. The same procedure was followed for each of the other acid gels prepared, and the initial pH values of the gel mixtures produced are shown in Table 2.

TABLE 2

| | Gel 1 | Gel 2 | Gel 3 | Gel 4 | Gel 5 |
|---|---|---|---|---|---|
| Sodium citrate content, | 0% | 1% | 3% | 10% | 15% |
| wt % | | | | | |
| Sodium citrate | 0M | 0.03M | 0.10M | 0.34M | 0.51M |
| Citric acid | 0.29M | 0.29M | 0.29M | 0.29M | 0.29M |
| Ascorbic acid | 0.30M | 0.30M | 0.30M | 0.30M | 0.30M |
| pH of acid gel | 1.59 | 2.4 | 3.12 | 4.41 | 4.48 |
| Initial pH of 2-gel | 3.59 | 3.70 | 4.28 | 4.79 | 4.94 |
| mixture | | | | | |

In the above compositions of Examples 2 and 2.1, the following acid dissociation reaction occurs:

HA [symbol] A⁻+H⁺

With a dissociation equilibrium constant:

$${K}_{a} = \frac{[ {A}^{-} ] [ {H}^{+} ]}{[ HA ]}$$

Where, in this case, HA is the citric acid and A⁻ is the citrate ion.

Addition of sodium citrate to the acid gel provides additional A⁻ and shifts the acid dissociation equilibrium so that the H⁺ concentration is reduced, increasing the pH of the acid gel. The further example of Example 2.1 shows that the addition of 15% sodium citrate reduces the amount of NO released by no more than 30% but raises the pH of the mixed product from 3.59 to 4.94, which is a reduction in its acidity (H⁺ concentration) by a factor of 22.

### EXAMPLE 3

### Experimental Procedure for Determining the NO Release from Mixed Gels

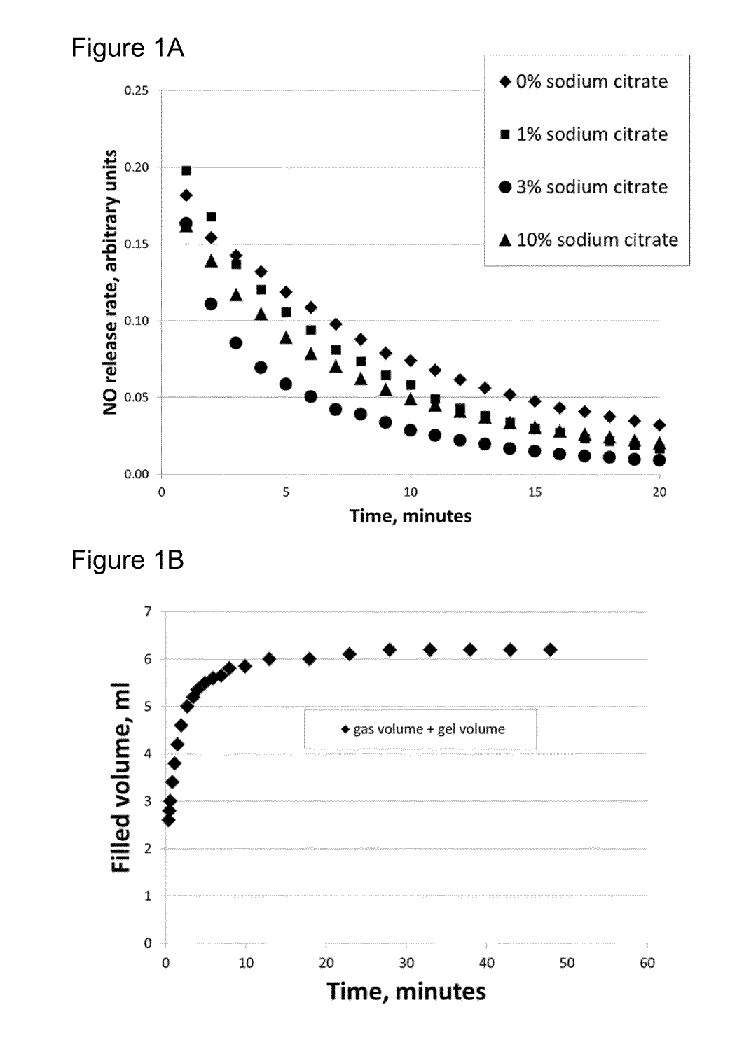

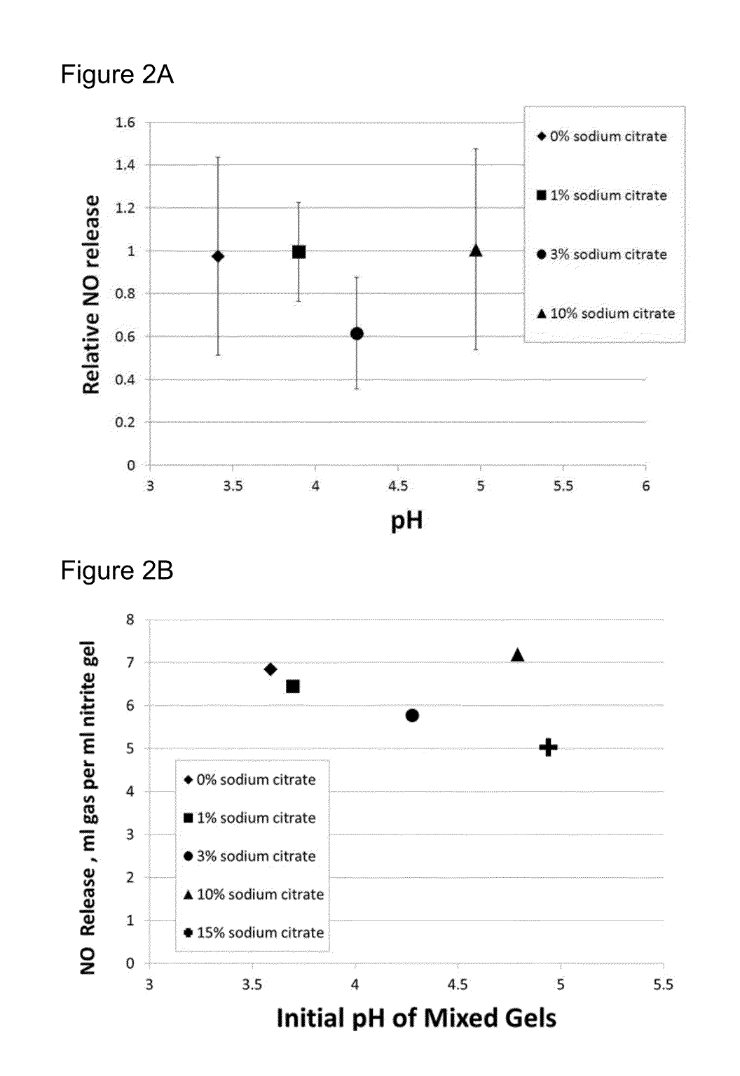

In Example 2, NO release was determined as follows. Equal amounts of acid-containing and nitrite-containing gels were mixed under a constant flow of Argon. NO evolving from the gel mixture was diluted by the Argon flow and the NO concentration in that flow was measured as a function of time. Measuring that concentration produced results shown in FIG. 1A for NO release rate from the gel mixture. The relative amounts of NO released by each gel mixture were determined by integration of the curves shown in FIG. 1A from time t=0 to time t=20 minutes to produce the results shown in FIG. 2A. Surprisingly, the inventors found the amounts of NO released from the gel mixtures containing various amounts of sodium citrate are not strongly dependent on the pH of the gel mixture in the pH range covered.

### EXAMPLE 3.1

### Experimental Procedure for Determining the NO Release from Mixed Gels

A simple method was employed to measure the total gas released by the gel mixtures using each of the five different gels of Example 2.1. Approximately 1.5 ml of each acid gel and an equal amount of nitrite gel were respectively loaded into opposite cylinders of a dual-cylinder, 2 ml mixing syringe fitted with a static mixer. The syringe was operated, and the gel mixture was dispensed directly into a small graduated cylinder. As soon as the gels are mixed, bubbles of NO begin to form in the gel. The gels mixture is sufficiently viscous that it forms a foam containing many bubbles of NO. As NO generation continues, the volume of the foam builds up in the graduated cylinder, and very little NO escapes from the foam through the top of the foam due to the small cross sectional area of the cylinder and the high viscosity of the gel. The volume of the foam in the graduated cylinder is measured throughout the time it increases and stabilizes. FIG. 1B shows the measured foam volume after injecting the mixture containing Gel 4 (10% sodium citrate) of Example 2.1 into the graduated cylinder. The foam is then allowed to remain in the graduated cylinder for approximately 16 hours, which is the time required for all the NO to escape, leaving a volume of bubble-free gel in the graduated cylinder. The volume of gas generated is thus given by the difference between the maximum foam volume observed (which is the gel volume plus the gas volume) and the bubble-free gel volume observed many hours later. The volumes of NO gas generated by the each of the gel compositions of Example 2.1 is shown in FIG. 2B, where the results are plotted as the volume of gas generated per ml of nitrite gel used. The data are highly consistent, in that the calculated stoichiometric NO release from the nitrite gel composition used is 6.5 ml of NO gas per ml of nitrite gel if the NO-producing reaction goes to completion. In all cases, the measured NO release is close to this value, indicating that the NO-producing reaction is going to completion. This data unambiguously shows that that there is substantial NO release from the mixed gel composition throughout the pH range between 3.5 and 5.

### EXAMPLE 4

### Demonstration of Increased Blood Flow Resulting from Topical Application of an Acidified Nitrite Gel Containing Sodium Citrate

Gels were prepared according to the mixing procedure of Example 1, but with amounts of the ingredients to achieve molarities of those ingredients as shown below. Euxyl® PE9010 is a preservative having phenoxyethanol as the active ingredient (Schülke and Mayr, 30 Two Bridges Road Suite 225, Fairfield, N.J. 07004, USA). 3-O-ethyl ascorbic acid, which is a derivative of ascorbic acid, was used instead of ascorbic acid because it was found that use of this compound preserves the color and viscosity of the composition over long periods of time.

Gel “N” for Nitric Oxide Donor Composition

| | | Weight | |
|---|---|---|---|
| Each ml contains: | | Percentage | Concentration |
| Sodium nitrite* | 5.0 mg | 0.50% | 73 mM |
| Euxyl ® PE9010 | 3.0 mg | 0.30% | |
| Hydroxyethylcellulose (HEC) | 28.8 mg | 2.88% | |
| 750000 | | | |
| Water to make 1 ml. | | | |

*In the case of the placebo formulation, the sodium nitrite above was replaced with 6.1 mg sodium bicarbonate, which is provides a concentration in the composition of 73 mM and a weight percentage of 0.61%. The placebo concentration will not release any NO when it is mixed with Gel “A”, the composition for which is shown below.

Gel “A” for Nitric Oxide Activation Composition:

| | | Weight | |
|---|---|---|---|
| Each ml contains: | | Percentage | Concentration |
| Citric acid | 14.0 mg | 1.40% | 73 mM |
| Sodium citrate | 4.7 mg | 0.47% | 18 mM |
| 3-O-ethyl ascorbic acid | 15.0 mg | 1.5% | 73 mM |
| Euxyl ® PE9010 | 3.0 mg | 0.30% | |
| Hydroxyethlycellulose (HEC) | 28.8 mg | 2.88% | |
| 750000 | | | |
| Water to make 1 ml. | | | |

The subjects were directed to lie flat on a bed and were asked to remain quiet and still throughout the duration of the study. The test skin areas (4 cm×5 cm=20 cm²) on the volar aspect of both forearms were marked with ink. Blood flux was measured using a dual channel Laser Doppler Perfusion Monitor (Moor DRT4) with probes applied to each forearm, taking care to avoid any superficial veins. The output of the Perfusion Monitor was continuously recorded by a laptop computer using custom software. After recording control measurements on both arms for about 5 minutes, the measurements were paused and the probes were taken away carefully. Each set of two gels were individually mixed in a one-to-one ratio (about 0.5 ml gel “N”+0.5 ml gel “A” for the active composition; and 0.5 ml placebo gel+0.5 ml gel “A” for the placebo composition) and applied to the marked area of each forearm. The placebo mixture was applied to the right arm, while the active ingredient mixture was applied to the left arm. After 1-2 min for air drying, the residual gel was removed and the probes were reattached to exactly the same position to continue the measurements.

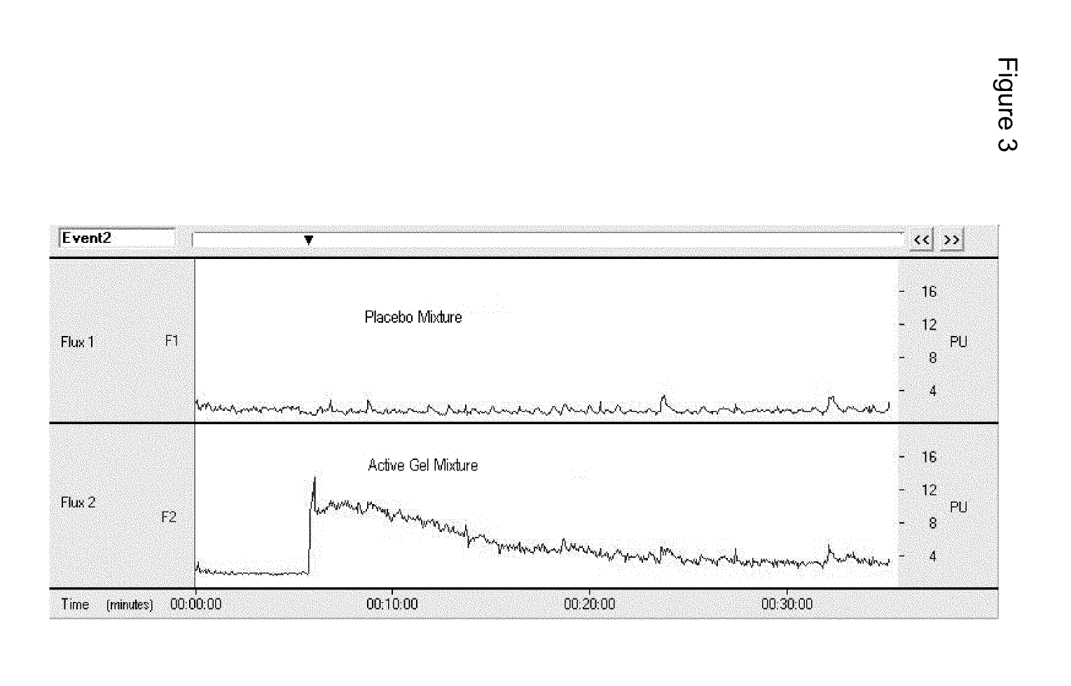

The measured perfusion in each test area is shown in FIG. 3, for both the active gel mixture and for the placebo gel mixture where the vertical arrowhead at the top of the figure indicates the time of application of the gels. The vertical axis corresponds to the relative blood perfusion measured. The blood perfusion increased immediately after applying the active ingredient gel. The perfusion initially decreased with a time constant of about 10 minutes, but remained elevated throughout the test. The placebo mixture appeared to have no effect on blood perfusion.

These tests indicate efficacy of gel treatments for enhancing blood circulation in areas where the treatment is applied. This is, however, just one of the applications of the technology. The present invention demonstrates that the systems described herein release NO, and also that they produce increased local blood flow. That observation certainly makes the discovery applicable in any case where increased local blood circulation can be therapeutic.

### EXAMPLE 5

### Applications of Gel Compositions

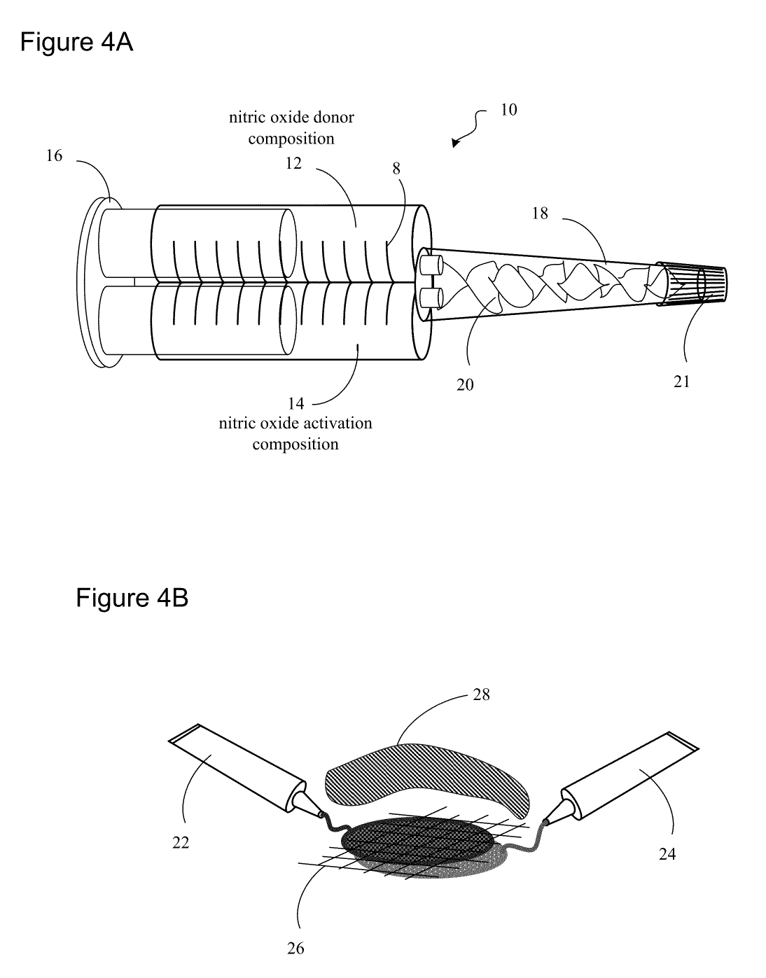

In one embodiment as depicted in FIG. 4A, an NO generation system is provided in a two chambered mixing apparatus such as mixing syringe 10. A nitric oxide donor composition including a nitrite salt in a gel is preloaded in first chamber 12 of mixing syringe 10. A nitric oxide activation composition including a reductant, at least one organic acid and at least one conjugate base of the organic acid mixed together in a gel that is preloaded in second chamber 14 f mixing syringe 10. The mixing apparatus is provided capped with cap 21 and the included compositions are stable for storage. When an NO generating admixture is desired, combined plunger 16 is pressed and the nitric oxide donor composition and nitric oxide activation compositions are expressed together through mixing chamber 18. In one embodiment, mixing chamber 18 includes a static mixing element 20 that adequately mixes the nitric oxide donor and activation compositions as the two compositions are expressed from the mixing apparatus.

The mixed compositions may be applied directly to a tissue site. In the depicted embodiment, the mixing apparatus is preloaded with a plurality of measured doses. A single dosage amount is provided with each depression of plunger 16 to each of single dosage markings 8. Because the nitric oxide donor and activator compositions are independently stable until admixed, when the mixing apparatus is prefilled with a plurality of doses, a repeated application can be made at such time as the prior dose has essentially exhausted its NO generation capacity. In the depicted embodiment for example, the mixing apparatus is preloaded with 12 doses that can be applied at intervals over a period such as a day. If desired the expressed admixed dose is covered with an occlusive bandage that prevents NO elaboration into the atmosphere and directs all NO produced to the tissue site. For each repeat dosage the bandage is lifted and a further dosage expressed under the bandage that is then lowered over the treated site. For example, if a new dosage is expressed from the mixing apparatus hourly, a tissue site can be exposed to NO coverage for a continuous 12 hour period. In certain embodiments, the prefilled mixing apparatus is provided in a wound healing kit that includes the prefilled mixing apparatus and the bandage in a sterile kit that includes instructions for use. In certain embodiments, a single compound such as an ascorbic acid or ascorbic acid derivative may function as both the reductant and the organic acid in the nitric oxide activating composition. In certain embodiments, the pH of the activation composition is greater than 3.0 and when admixed with the nitric oxide donor, the admixture is characterized by an initial pH greater than 4.0, thus minimizing pain on application.

In another embodiment as depicted in FIG. 4B, NO is produced locally by first applying a gel form nitric oxide donor composition the skin or other local body site 26. This would be followed by overlaying a nitric oxide activation composition layer comprising acid, reductant and base. This should give rise to a controlled rate of nitric oxide release to the desired bodily surface. As depicted, the nitric oxide donor composition is supplied in and expressed from tube or other container 24 to the site. The nitric oxide activation composition is supplied in and expressed from tube or other container 22 to the site overlying the application of the nitric oxide donor composition. The tube or other containers are prefilled with premeasured amounts. If desired the layers are covered with an occlusive bandage 28 that directs all NO produced to the tissue site.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A biocompatible two-component nitric oxide (NO) delivery system that comprises:
a nitric oxide donor composition comprising a nitrite salt in a first gel, and
a nitric oxide activation composition comprising at least one reductant, at least one organic acid and at least one conjugate base of the organic acid mixed together in a second gel, wherein the pH of the activation composition is greater than 3.0,
wherein the nitric oxide donor composition and nitric oxide activation composition are stable when stored separately and generate a source of nitric oxide when admixed, the admixture characterized by an initial pH greater than 4.0.

2. The system of claim 1, wherein the nitrite salt is selected from one or more of the group consisting of: sodium nitrite, potassium nitrite, and calcium nitrite.

3. The system of claim 1, wherein the organic acid is selected from one or more of the group consisting of: citric acid, ascorbic acid, lactic acid, glyceric acid, and formic acid.

4. The system of claim 1, wherein the organic acid is ascorbic acid and the ascorbic acid further functions as the at least one reductant.

5. The system of claim 1, wherein the organic acid is an ascorbic acid derivative and the ascorbic acid derivative further functions as the at least one reductant.

6. The system of claim 5, wherein the ascorbic acid derivative is selected from the group consisting of:
3-O-ethyl ascorbic acid, other 3-alkyl ascorbic acids, 6-O-octanoyl-ascorbic acid, 6-O-dodecanoyl-ascorbic acid, 6-O-tetradecanoyl-ascorbic acid, 6-O-octadecanoyl-ascorbic acid, 6-O-dodecanedioyl-ascorbic acid and combinations thereof

7. The system of claim 5, wherein the ascorbic acid derivative is 3-O-ethyl ascorbic acid.

8. The system of claim 1 wherein the nitric oxide activation composition comprises a citric acid, a sodium citrate and an ascorbic acid or ascorbic acid derivative.

9. The system of claim 1, wherein the first and second gels comprise hydroxyethylcellulose.

10. The system of claim 1, wherein the nitric oxide donor composition and the nitric oxide activation composition are provided pre-loaded in a two chambered mixing apparatus.

11. The system of claim 10 wherein the two chambered mixing apparatus further includes a static mixer.

12. The system of claim 1, wherein the nitric oxide donor composition and the nitric oxide activation composition are provided in two separate containers prefilled with pre-measured quantities of the donor and activation compositions and adapted to deliver the pre-measured quantities of the donor and activation compositions to a tissue site.

13. The system of claim 1, further comprising an occlusive bandage packaged together with the nitric oxide donor and activation compositions.

14. A two-component nitric oxide (NO) delivery system that comprises:
a first chamber including a plunger and containing a nitric oxide donor composition comprising a nitrite salt in a first gel, and
a second chamber including a plunger and containing a nitric oxide activation composition comprising at least one reductant, at least one organic acid and at least one conjugate base of the organic acid mixed together in a second gel,
wherein the first chamber and second chambers are adapted store the nitric oxide donor and activation compositions separately until nitric oxide generation is desired at which time the plungers are adapted to be moved synchronously into the first and second chambers and thereby extrude measured amounts of the nitric oxide donor and activation compositions into a mixing chamber that is adapted to admix the measured amounts and extrude a nitric oxide generating admixture to a tissue site.

15. The system of claim 14, wherein the first and second chambers are fixed together as two separate chambers of a syringe.

16. The system of claim 14, wherein the mixing chamber includes a static mixer that is adapted to adequately mix the nitric oxide donor and activation compositions as the two compositions are expressed from the delivery system.

17. The system of claim 14, wherein the first and second chambers are preloaded with a plurality of single doses.

18. The system of claim 17, wherein the chambers are marked with single dosage markings that indicate each single dosage amount to be delivered with each depression of the plungers.

19. The system of claim 14 further comprising an occlusive bandage packaged together with the nitric oxide donor and activation compositions disposed in the first and second chambers.

20. The system of claim 14, wherein the pH of the nitric oxide activation composition is greater than 3.0 and the admixture is characterized by an initial pH greater than 4.0.

21. The system of claim 14, wherein the organic acid is an ascorbic acid derivative and the ascorbic acid derivative further functions as a reductant.

22. The system of claim 21, wherein the ascorbic acid derivative is selected from the group consisting of:
3-O-ethyl ascorbic acid, other 3-alkyl ascorbic acids, 6-O-octanoyl-ascorbic acid, 6-O-dodecanoyl-ascorbic acid, 6-O-tetradecanoyl-ascorbic acid, 6-O-octadecanoyl-ascorbic acid, 6-O-dodecanedioyl-ascorbic acid and combinations thereof

23. The system of claim 14, wherein the nitrite salt is selected from one or more of the group consisting of: sodium nitrite, potassium nitrite, and calcium nitrite.

24. A biocompatible two-component nitric oxide (NO) delivery system that comprises:
a nitric oxide donor composition comprising a nitrite salt at a fixed molarity, and
a nitric oxide activation composition comprising a citric acid and an ascorbic acid or a ascorbic acid derivative , both acids at essentially the same molarity as the nitrite salt, and further comprising sodium citrate at a concentration sufficient to increase the nitric oxide activation composition to a pH greater than 3.0,
wherein the nitric oxide donor composition and nitric oxide activation composition are stable when stored separately and generate a source of nitric oxide when admixed, the admixture characterized by an initial pH greater than 4.0.

25. The biocompatible two-component nitric oxide (NO) delivery system of claim 24 wherein the nitric oxide donor composition and the nitric oxide activation composition are provided in a gel form.

26. The biocompatible two-component system of claim 24, wherein the ascorbic acid derivative is selected from the group consisting of:
3-O-ethyl ascorbic acid, other 3-alkyl ascorbic acids, 6-O-octanoyl-ascorbic acid, 6-O-dodecanoyl-ascorbic acid, 6-O-tetradecanoyl-ascorbic acid, 6-O-octadecanoyl-ascorbic acid, 6-O-dodecanedioyl-ascorbic acid and combinations thereof

27. The biocompatible two-component system of claim 24, wherein the nitrite salt is selected from one or more of the group consisting of: sodium nitrite, potassium nitrite, and calcium nitrite.

28. The biocompatible two-component nitric oxide (NO) delivery system of claim 24 wherein the nitric oxide donor composition and nitric oxide activation composition are provided in premeasured doses in separate containers that are adapted to deliver the nitric oxide donor and activation compositions in suitable admixture amounts to generate a prolonged release of nitric oxide when admixed.
